(19) United States
(12) Patent Application Publication
(10) Pub. No.: US 2025/0288307 A1
(43) Pub. Date: Sep. 18, 2025

(54) INTRAVASCULAR/ENDOVASCULAR ULTRASOUND TRANSDUCERS CAPABLE OF GENERATING SWIRLING ACOUSTIC FIELD

(71) Applicants: North Carolina State University, Raleigh, NC (US); Georgia Institute of Technology, Atlanta, GA (US)

(72) Inventors: Xiaoning Jiang, Cary, NC (US); Chengzhi Shi, Marietta, GA (US); Howuk Kim, San Jose, CA (US); Bohua Zhang, Raleigh, NC (US); Huaiyu Wu, Raleigh, NC (US)

(21) Appl. No.: 18/859,004

(22) PCT Filed: Apr. 21, 2023
(86) PCT No.: PCT/US2023/019428

§ 371 Date: Oct. 22, 2024

Related U.S. Application Data
(60) Provisional application No. 63/333,731, filed on Apr. 22, 2022.

(51) Int. Cl. A61B 17/22; A61B 17/00; A61M 5/00

(52) U.S. Cl. CPC A61B 17/2202; A61M 5/007; A61B 2017/00402; A61B 2017/22028; A61B 2017/22082

(57) ABSTRACT

The present invention relates to vortex ultrasound transducers and methods of therapeutic ultrasound treatment. A device for therapeutic ultrasound treatment includes a jacket for insertion within a blood vessel of a patient and an ultrasound transducer located within the jacket and comprising a plurality of active elements that are arranged such that, when activated, the ultrasound transducer is configured to generate ultrasound energy with a swirling movement that produces a mechanical shear stress over a target area within the blood vessel of the patient.

### PRIORITY CLAIM

This application claims the priority benefit of U.S. Provisional Patent Application Ser. No. 63/333,731 entitled “INTRAVASCULAR/ENDOVASCULAR ULTRASOUND TRANSDUCERS CAPABLE OF GENERATING SWIRLING ACOUSTIC FIELD,” filed on Apr. 22, 2022, the disclosure of which is incorporated herein by reference in its entirety.

### GOVERNMENT SUPPORT

This invention was made with government support under grant numbers EB027304, HL141967, and HL154735 awarded by the National Institutes of Health. The government has certain rights in the invention.

### TECHNICAL FIELD

The subject matter described herein relates to ultrasound transducers. More specifically, the subject matter relates to vortex ultrasound (VUS) transducers and methods for producing such.

### BACKGROUND

Known therapeutic ultrasound applications are primarily transcutaneous in nature and, since the focal point of the ultrasound generated is internal to the body to which the treatment is being applied, can result in unwanted damage to tissues that are adjacent to the treatment site. While such noninvasive treatment modalities may be used in some instances for treatment of, for example, intravascular blood clots, the inability to precisely visualize and, thereby, control the target of the ultrasound can lead to such unwanted damage to surrounding tissues. Furthermore, some such blood clots may be located within a blood vessel that is not adjacent to the skin and, as such, use of a conventional noninvasive ultrasound transducer may be impractical and/or impossible.

Furthermore, even conventional intravascular ultrasound transducers only generate a single-phase acoustic pressure output, where the stress gradient at the target region and the associated shear stress is weak. Therefore, increasing the acoustic pressure output using such a conventional intravascular ultrasound transducer is typically the only way to increase the treatment speed in intravascular/endovascular ultrasound therapies. This is a known limitation of such intravascular ultrasound transducers, in which it has traditionally been very difficult from a technical perspective to intensify the acoustic pressure output, as well as the resulting cavitation intensity, due to the geometric constraints for such transducers, which are generally limited to having a diameter in the range of about 5-6 Fr. (French gauge size) (or 1.67-2 mm). Thus, in such conventional approaches, the only way to transmit a high acoustic pressure output to a target region is to increase the input voltage level of the transducer. The presently disclosed subject matter addresses the limitations associated with these conventional intravascular/endovascular ultrasound transducers, allowing for improved operational (e.g., therapeutic) efficacy without requiring such substantially increased input voltage levels and operational frequencies.

### SUMMARY

Devices for therapeutic ultrasound treatment and methods of therapeutic ultrasound treatment are disclosed. An example device for therapeutic ultrasound treatment includes a jacket for insertion within a blood vessel of a patient and an ultrasound transducer located within the jacket and comprising a plurality of active elements that are arranged such that, when activated, the ultrasound transducer is configured to generate ultrasound energy with a swirling movement that produces a mechanical shear stress over a target area within the blood vessel of the patient.

An example method of therapeutic ultrasound treatment includes providing a device for therapeutic ultrasound treatment. The device includes a jacket for insertion within a blood vessel of a patient and an ultrasound transducer located within the jacket and comprising a plurality of active elements that are arranged such that, when activated, the ultrasound transducer is configured to generate ultrasound energy with a swirling movement. The method further includes positioning the device in a position adjacent to a target area designated for receiving the therapeutic ultrasound treatment. The method further includes emitting, from the ultrasound transducer, the ultrasound energy with the swirling movement at the target area, the ultrasound energy producing a mechanical shear stress over a target area within the blood vessel of the patient.

In some embodiments, the jacket may include a catheter, wherein the catheter comprises a flow channel configured to inject one or more contrast agents within a field of the ultrasound energy emitted from the ultrasound transducer. Each of the plurality of active elements may be a piezoelectric element. The plurality of active elements may be sequentially activated in a clockwise direction or a counterclockwise direction, with respect to a longitudinal axis of the ultrasound transducer. The plurality of active elements may have sequentially greater height in a clockwise direction or a counterclockwise direction, with respect to a longitudinal axis of the ultrasound transducer. The plurality of active elements may have approximately a same height. The device may include a circuit connected to the plurality of active elements, wherein the circuit is configured to generate a signal targeting one or more select active elements of the plurality of active elements. The circuit may be configured to sequentially transmit a signal to one or more select active elements of the plurality of active elements in a clockwise direction or a counterclockwise direction, with respect to a longitudinal axis of the ultrasound transducer. The circuit may be configured to reverse the direction of the sequential transmission. The target area may be a blood clot.

### BRIEF DESCRIPTION OF THE DRAWINGS

The subject matter described herein will now be explained with reference to the accompanying drawings of which:

FIG. 1 is a schematic diagram illustrating an example use of a VUS transducer according to an embodiment of the presently disclosed subject matter;

FIG. 2 is a schematic diagram of a plurality of ultrasound emitter elements according to an embodiment of the presently disclosed subject matter;

FIG. 3 is a schematic diagram illustrating groups of piezoelectric stacks according to an embodiment of the presently disclosed subject matter;

FIG. 4 is a perspective view of a piezoelectric stack according to an embodiment of the presently disclosed subject matter;

FIG. 5A is a schematic diagram of a VUS transducer with an electric phase-delaying method according to an embodiment of the presently disclosed subject matter;

FIG. 5B is a schematic diagram of a VUS transducer with a mechanical phase-delaying method according to an embodiment of the presently disclosed subject matter;

FIG. 6A is a plot illustrating acoustic pressure phase angle from the aperture surface of a non-vortex ultrasound (i.e., conventional) transducer;

FIG. 6B is a plot illustrating acoustic pressure phase angle from a VUS transducer according to an embodiment of the presently disclosed subject matter; and

FIG. 7 is an image and front end view of an example embodiment of a VUS transducer according to an embodiment of the presently disclosed subject matter;

FIG. 8 is a bar chart illustrating the efficacy of using a VUS transducer for thrombolysis in-vitro with bovine blood clots according to an embodiment of the presently disclosed subject matter;

FIG. 9A is a plot of the amplitude field of a VUS transducer according to an embodiment of the presently disclosed subject matter;

FIG. 9B is a plot of the phase pattern generated by a VUS transducer according to an embodiment of the presently disclosed subject matter;

FIG. 9C is a plot of the amplitude field of a transducer array with a flat forward viewing surface;

FIG. 9D is a plot of the phase pattern generated by a transducer array with a flat forward viewing surface;

FIG. 10 is a flow chart of an example method of manufacturing a VUS transducer according to an embodiment of the presently disclosed subject matter;

FIG. 11 is a flow chart of an example method of manufacturing a VUS transducer according to an embodiment of the presently disclosed subject matter; and

FIG. 12 is a flow diagram illustrating example method of therapeutic ultrasound treatment according to an embodiment of the presently disclosed subject matter.

### DESCRIPTION

The presently disclosed subject matter now will be described more fully hereinafter, in which some, but not all embodiments of the presently disclosed subject matter are described. Indeed, the presently disclosed subject matter can be embodied in many different forms and should not be construed as limited to the embodiments set forth herein; rather, these embodiments are provided so that this disclosure will satisfy applicable legal requirements.

Disclosed herein is an intravascular vortex ultrasound (VUS) transducer, as well as associated therapeutic treatment systems and methods of using such an VUS transducer. Such VUS transducers, systems, and methods disclosed herein are, amongst other uses, advantageously suited for use in lysing tumors and blood clots, as well as for localized drug delivery (e.g., at the site of a tumor, blood clot, etc.). Using such VUS transducers in intravascular/endovascular therapeutic techniques advantageously enables precise delivery of the ultrasound beam to the target, which reduces damage to surrounding tissues that are known to occur when using conventional transcutaneous ultrasound devices and techniques.

The VUS transducers disclosed herein comprise active elements, such as piezoelectric stacks with multiple piezoelectric elements, as well as a phase delaying mechanism, such that the VUS transducers can, via the ultrasound acoustic energy emitted from the active elements, cause mechanical shear stress over a target region in addition to the cavitation effect and radiation forces generated by conventional ultrasound transducers. By inducing a mechanical shear stress over the target region, such VUS transducers are capable of decreasing the time required for therapeutic ultrasound treatment. Furthermore, the swirling movement of the ultrasound generated by such VUS transducers also allows for the intravascular VUS transducers to have a lower power consumption compared to conventional non-swirling ultrasound transducers, while still providing the same clinical outcome as conventional non-swirling ultrasound transducers. Among other uses, the VUS transducers can be used for rapid debulking of a blood clot within a blood vessel (e.g., venous or arterial blood clots) on which a conventional ultrasound transducer is generally incapable of therapeutic operation.

The VUS transducers disclosed are particularly advantageously used in intravascular/endovascular therapeutic treatments, for example lysing tumors and blood clots, as well as for localized drug delivery. When used in such intravascular/endovascular therapeutic treatments, the VUS transducers can be used to precisely deliver the ultrasound beam to the target, reducing unwanted tissue damages associated with conventional transcutaneous ultrasound therapeutic treatments. Although the present disclosure describes embodiments where the VUS transducers include piezoelectric ultrasound transducers, the VUS transducers are not limited to this type of transducer. The VUS transducers disclosed may include, for example, piezoelectric ultrasound transducers, optical ultrasound transducers, acoustic ultrasound transducers, or any other ultrasound transducers known in the art consistent with this disclosure. In embodiments wherein the VUS transducer includes a piezoelectric ultrasound transducer, the active elements may be piezoelectric elements. The piezoelectric ultrasound transducer may include piezoelectric stacks each with one or more of the piezoelectric elements.

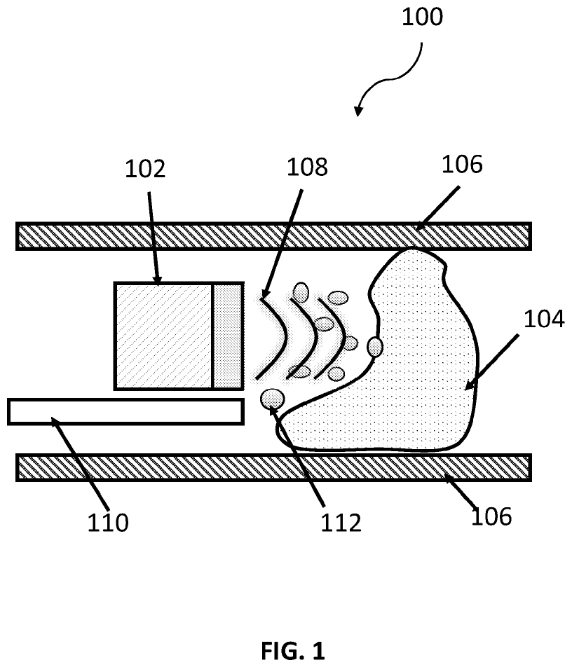

One example use of such a VUS transducer for therapeutic treatments is for intravascular or endovascular sonothrombolysis of venous or arterial blood clots. FIG. 1 is a schematic diagram illustrating a system 100 of an example use of a miniaturized VUS transducer 102 performing intravascular thrombolysis. According to this example embodiment, VUS transducer 102 is positioned adjacent to a target region (e.g., where a blood clot 104 is located) within a blood vessel 106. VUS transducer 102 generates an ultrasound beam 108 of acoustic energy onto and/or over the target region including blood clot 104. VUS transducer 102 can emit low frequency ultrasound, for example less than 2 Hz. In the example embodiment in which blood clot 104 is within the target region, the mechanical cavitation induced by VUS transducer 102 dissolves the fibrin structures of the blood clot 102. Furthermore, VUS transducer 102 may be integrated with a catheter that has a flow channel 110 formed along the length thereof for the delivery of, for example, cavitation agents 112 such as microbubbles (MBs) and/or nanodroplets (NDs) to a location directly adjacent to blood clot 104, which can advantageously expedite the lysis process by reducing the cavitation threshold.

As will be explained further herein, VUS transducer 102 shown in FIG. 1 causes a swirling movement in the fluid flow; the swirling movement is induced due to the novel manner in which the ultrasonic acoustic energy is generated, such that the VUS transducer 102 can provide mechanical agitation as well as a cavitation effect. This swirling component (e.g., in the manner of a vortex) induced in the fluid flow causes mechanical shear stress over target regions (e.g., a blood clot). Since a conventional ultrasound transducer exhibits only a single phase of acoustic pressure output at the targeted region, such a complex, swirling movement of the fluid flow cannot be generated using such conventional ultrasound transducers. In contrast, according to the subject matter disclosed herein, a multi-phased acoustic pressure output can be realized, such that the swirling movement of the fluid flow is provided in addition to the localized cavitation effects provided by conventional ultrasound transducers. Providing such a multi-phase acoustic output pressure is thus heretofore unknown using such conventional ultrasound transducers for intravascular/endovascular therapeutic ultrasound applications.

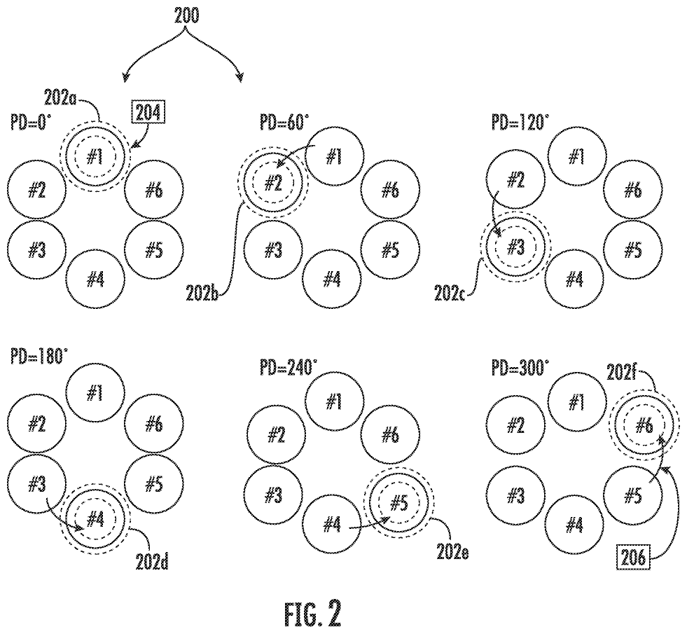

FIG. 2 is a schematic illustration of a plurality of ultrasound emitter elements such as piezoelectric stacks 202 that, together, form a VUS transducer 200 that can generate, or induce, a swirling movement 206 in a fluid flow, according to an example embodiment disclosed herein. VUS transducer 200 can include a plurality of active elements configured to generate ultrasound energy. VUS transducer 200 can include stacks wherein each of the stacks have one or more of the plurality of active elements. As shown in FIG. 2, VUS transducer 200 is an example embodiment including a piezoelectric ultrasound transducer. VUS transducer 200 comprises a plurality of stacks, such as piezoelectric stacks 202, each with active elements, such as piezoelectric elements, which are operated with different phases. In the example shown in FIG. 2, VUS transducer 200 comprises a quantity of six (6) piezoelectric stacks 202 that are arranged in a generally annular shape. Regardless of the quantity of piezoelectric stacks 202 that form VUS transducer 200, the phase at which each piezoelectric stack 202 is activated to generate swirling movement 206 in the fluid flow is determined by dividing 360° by the quantity of piezoelectric stacks 202.

In the example embodiment shown in FIG. 2, in which VUS transducer 200 comprises six (6) piezoelectric stacks 202, the phase delay (PD) between activation of piezoelectric stacks (e.g., adjacent piezoelectric stacks) is 60°. The quantity of piezoelectric stacks 202 is not limited to that which is shown in FIG. 2, however, and can include more than or less than six (6); however, it is contemplated that the quantity of piezoelectric stacks 202 must be at least three (3) in order to induce swirling movement 206 to the fluid flow.

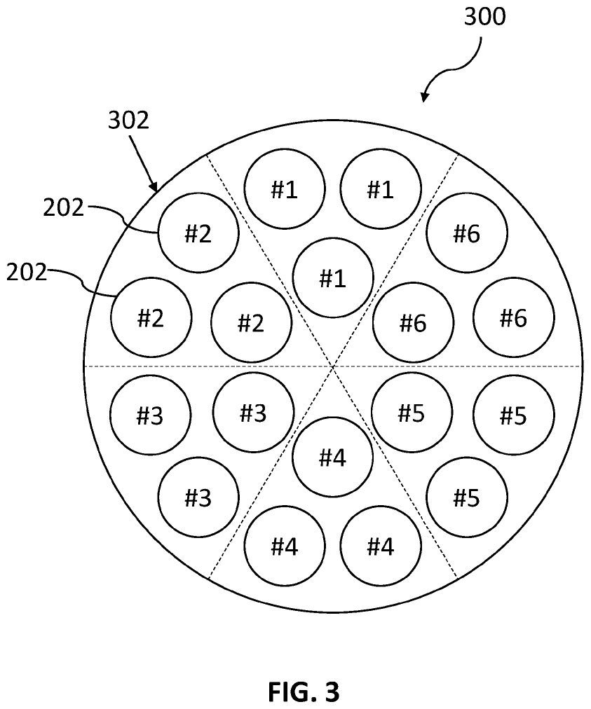

FIG. 3 shows a schematic diagram of an example embodiment including groups 302 of piezoelectric stacks 202. Referring now to FIG. 3, in some embodiments, stacks of VUS transducer 300, such as piezoelectric stacks 202 Illustrated in FIG. 3, may be grouped together, such that more than one active element, e.g., piezoelectric stack 202, is activated at the same time. In some such embodiments, piezoelectric stacks 202 may be arranged in radially adjacent rings, for example, each group 302 having a plurality of piezoelectric elements. Thus, each group 302 comprises three (3) piezoelectric stacks 202 in a VUS transducer 300 shown in FIG. 3. The quantity of piezoelectric stacks 202 in each group 302 can be the same or different. Furthermore, while piezoelectric stacks 202 that are in the same group 302 are typically physically adjacent to (e.g., contiguous with) each other, in some embodiments piezoelectric stacks 202 of the same group 302 may be spaced circumferentially (e.g., along the generally annular shape of the piezoelectric stacks) from each other. In some such instances, in which there are at least four (4) piezoelectric stacks 202, diametrically opposing piezoelectric stacks 202 may be grouped together.

Returning to the example embodiment shown in FIG. 2, at PD=0°, a first piezoelectric stack 202a is activated and a first ultrasound pulse (UP) 204 is emitted from the first piezoelectric stack 202a. After waiting a predefined phase delay, a second piezoelectric stack 202b, which is adjacent (e.g., directly) to first piezoelectric stack 202a, is activated and a second ultrasound pulse 204 is emitted from the second piezoelectric stack 202b. After waiting another phase delay, a third piezoelectric stack 202c, which is adjacent (e.g., directly) to second piezoelectric stack 202b, is activated and a third ultrasound pulse 204 is emitted from the third piezoelectric stack 202c. This process is repeated sequentially for a fourth piezoelectric stack 202d, a fifth piezoelectric stack 202e, and a sixth piezoelectric stack 202f until the phase delay becomes 360°, at which time first piezoelectric stack 202a is activated to generate the next pressure output. In the example embodiment of FIG. 2, since there are six (6) piezoelectric stacks 202 shown therein, the phase delay for activation of each subsequent piezoelectric element (e.g., after activation of an immediately previous piezoelectric stack) is 60°. According to the use of a phase delay for providing sequential activation of the piezoelectric stacks (e.g., the activation order proceeding in a circumferential direction of the generally annular shape), the rotational nature of the emission of acoustic pressure output from VUS transducer 200 induces swirling movement 206 (e.g., in the direction indicated by SW) in the fluid flow of a fluidic media (e.g., blood, water, etc.). This swirling movement 206 of the fluid flow and the shear stress associated therewith can thus advantageously be transferred to the target region. In instances in which it is desirous to not produce a fluid flow having swirling movement 206, one or more (e.g., some or all) piezoelectric stacks 202 can be activated substantially in unison. The quantity of piezoelectric stacks 202 that are activated substantially in unison can be used to alter an intensity of the acoustic pressure output of VUS transducer 200. This is also true for embodiments in which multiple piezoelectric stacks 202 are grouped together for simultaneous activation to induce a fluid flow with swirling movement 206, in which case a lower intensity for the acoustic pressure output can be selected by activating less than all (e.g., only one) piezoelectric stacks 202 in each group 302 and a high intensity for the acoustic pressure output can be selected by increasing the quantity of the piezoelectric stacks 202 that are activated in each group 302, as shown in FIG. 3. In embodiments having, for example, four (4) piezoelectric stacks 202, the phase delay for activation of a subsequent piezoelectric stack 202 after activation of an immediately previous piezoelectric stack would be 90°. In embodiments having, for example, ten (10) piezoelectric stacks 202, the phase delay for activation of a subsequent piezoelectric stack 202 after activation of an immediately previous piezoelectric stack 202 would be 36°. In the example embodiment shown in FIG. 2, the phase delay between activation of each of the piezoelectric stacks 202 after activation of an immediately previous piezoelectric stack is the same. However, the subject matter is not limited thereto; thus, in some embodiments, the phase delay between activation of each of the piezoelectric stacks 202 after activation of an immediately previous piezoelectric stack 202 may be different for some or all of the piezoelectric stacks 202. Merely by way of example, using the arrangement of piezoelectric stacks 202 shown in FIG. 2, it is thus envisioned that the phase delay for activation of second piezoelectric stack 202b could be 45°, the phase delay for activation of third piezoelectric stack 202c could be 135°, the phase delay for activation of fourth piezoelectric stack 202d could be 180°, the phase delay for activation of fifth piezoelectric stack 202e could be 270°, and the phase delay for activation of sixth piezoelectric stack 202f could be 330°. Thus, the phase delay associated with activation of any of piezoelectric stacks 202 can be any suitable value.

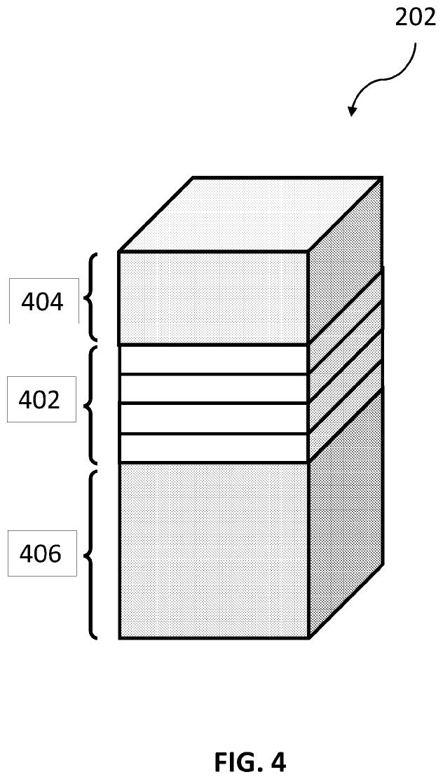

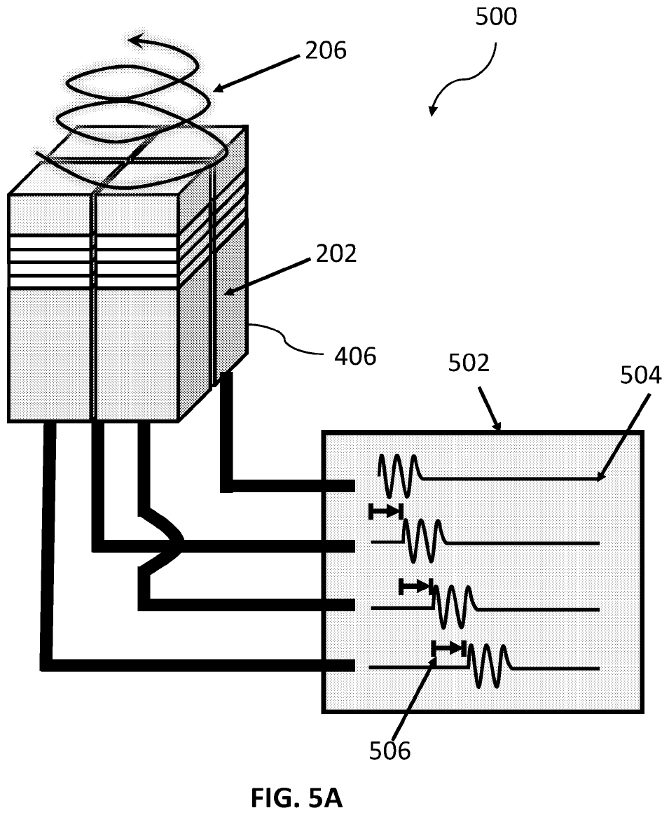

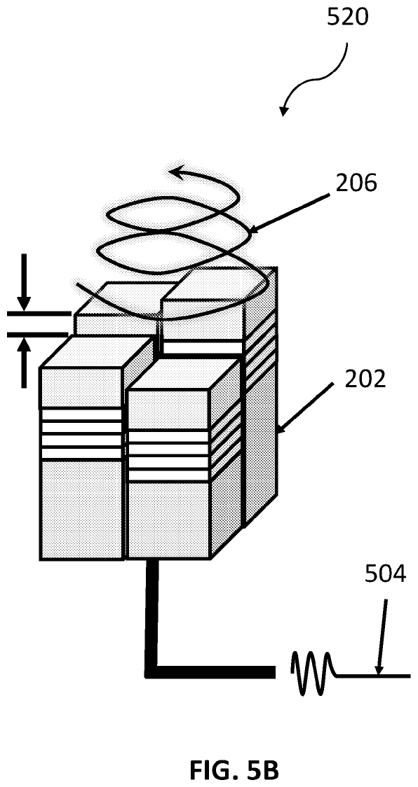

FIG. 4 is a perspective view of an example embodiment of piezoelectric stack 202 suitable for use in VUS transducers disclosed herein. As described herein, VUS transducers can include active elements, such as piezoelectric elements, which may be within stacks, such as piezoelectric stacks. According to the example embodiment, piezoelectric stack 202 comprises piezoelectric elements 402 that are, for example, stacked on top of each other in a sequential manner, a matching layer 404, and a backing layer 406. Alternative poling directions are applied between adjacent piezoelectric layers in piezoelectric elements 402. When an electric current is provided to piezoelectric stack 202, piezoelectric elements 402 of the piezoelectric stack 202 mechanically vibrate to produce ultrasound acoustic energy (e.g., waves) due to the piezoelectric effect. The ultrasound acoustic energy is further intensified within matching layer 404, having a quarter wavelength, due to the acoustic matching with the counterpart acoustic media (e.g., blood, water, etc.). Backing layer 406 is provided to suppress backpropagation of the ultrasound acoustic energy and, as such, the backing layer 406 advantageously comprises, consists of, or consists essentially of a material having a low acoustic impedance (e.g., about 1000 Rayls or less). FIG. 5A shows a schematic diagram of a VUS transducer 500 with an electric phase-delaying method. VUS transducer 500 includes piezoelectric stacks 202 connected to a phase delaying circuit 502. Each of the plurality of piezoelectric stacks 202 (or groups of piezoelectric stacks) receives a signal, such as an input waveform 504, via phase delaying circuit 502 and transmits an ultrasound pulse based on when the input waveform 504 is received by each piezoelectric stack 202 (or group thereof). As shown in FIG. 5A, input waveforms 504 each have a different phase delay 506 from each other, thereby inducing the fluid flow to have swirling movement 206 about the axial direction (e.g., parallel to the blood vessel axis) due to the sequential activation of piezoelectric stacks 202 in a generally annular, or circumferential, direction.

FIG. 5B shows a schematic diagram of another example embodiment of a VUS transducer 520 with a mechanical phase-delaying method, in which the effective phase delay between activation of piezoelectric stacks 202 can be controlled mechanically by selecting the height of the aperture surface(s) of each of the piezoelectric stacks 202 of the VUS transducer 520. Each of piezoelectric stacks 202 of VUS transducer 520 may be substantially similar to the construction of example piezoelectric stack 202 shown in FIG. 4. Unlike VUS transducer 500 shown in FIG. 5A, VUS transducer 520 does not require independent electric circuits for each piezoelectric stack 202 and the wiring of the piezoelectric stacks 202 can be made comparatively simpler via use of a single input (e.g., a coaxial cable) transmitting a single input waveform 504 to all of piezoelectric stacks 202 of the VUS transducer 520. In this example embodiment, VUS transducer 520 comprises a plurality of piezoelectric stacks 202 with a height difference 522. In some embodiments, piezoelectric stacks 202 can be grouped together, such as is shown in FIG. 3, with each piezoelectric stack 202 in the same group having substantially the same (e.g., identical) height.

In the example embodiment shown in FIG. 5B, all piezoelectric stacks 202 have a height difference 522 from each other. The height differences 522 among piezoelectric stacks 202 is selected according to the relationship of wavelength divided by the quantity of piezoelectric stacks 202. Height difference 522 between piezoelectric stacks 202 can be created by lapping (e.g., reducing) the thickness of backing layer 406 of each respective piezoelectric stack 202, such that the quantity, size, and/or shape of piezoelectric elements 402 and of matching layer 404 are substantially the same (e.g., identical, accounting for manufacturing tolerances) for all of the piezoelectric stacks 202, or by embedding the piezoelectric stacks 202 on a base (e.g., a 3D printed plastic base) that satisfies the required height difference (or phase delay) between each piezoelectric stack 202.

The mechanical phase delay embodiment of VUS transducer 520 shown in FIG. 5B is advantageous in terms of minimization of electric circuitry and wiring spots, but the resulting acoustic beam pattern has been found to suffer from a slight decrease in homogeneity compared to the electrical phase delay embodiment shown in FIG. 5A. This slight decrease in homogeneity is due in large part to the geometric unevenness of the aperture heights of each of the piezoelectric stacks. Moreover, it has been found that the use of electric control for phase delay can be advantageous because such electrically-controlled VUS transducers, such as VUS transducer 500 shown in FIG. 5A, can generate different swirling flow patterns based on input waveforms 504 and the sequence in which the input waveforms 504 are transmitted to each of the piezoelectric stacks. For example, by reversing the order in which input waveforms 504 are transmitted to piezoelectric stacks 202, the direction of the swirling aspect of the fluid flow induced by electrically-controlled VUS transducer 500 illustrated in FIG. 5A can also be reversed; such reversal in the direction of the swirling aspect of the fluid flow is not possible using mechanically-controlled VUS transducer 520 illustrated in FIG. 5B.

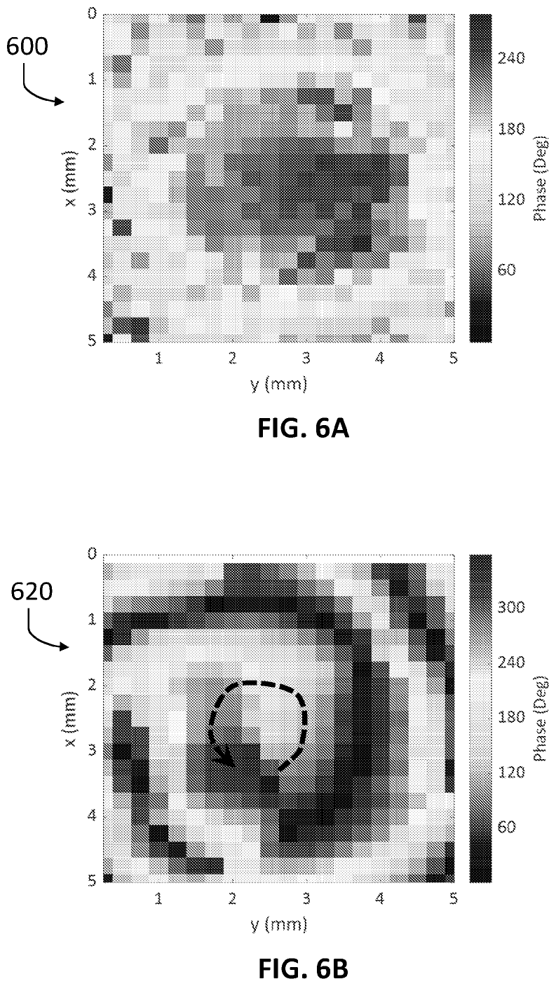

FIGS. 6A and 6B illustrate acoustic pressure phase angle captured at a distance of 1.5 wavelengths from the aperture surface of a non-swirl flow ultrasound (i.e., conventional) transducer in plot 600 and VUS transducer in plot 620, respectively. Plot 620 produced using a VUS transducer, shown in FIG. 6B, clearly shows phase variation in the swirling pattern of the ultrasound acoustic energy produced. Plot 600 produced using the conventional ultrasound transducer, shown in FIG. 6A, however, clearly shows a significant reduction in phase variation and, furthermore, also does not exhibit any swirling pattern in the plot of acoustic pressure.

FIG. 7 is an image and front end view of an example embodiment of a VUS transducer 700 with flow channel 110 (shown in FIG. 1). VUS transducer 700 is positioned (e.g., embedded) within a flexible catheter 702. Catheter 702 can include a first lumen 704 and a second lumen 706, each of which extends along the length of the catheter 702 and are substantially parallel to each other. First lumen 704 of catheter 702 is used for guiding VUS transducer 700, while second lumen 706 of the catheter 702 is used as a flow channel 110 for injection of a flow of, for example, a fluidic medium, such as contrast agents, within the field of the ultrasound acoustic energy produced by the VUS transducer 700. Flow channel 110 can include a longitudinally-extending flow channel for allowing introduction of a fluidic medium via VUS transducer 700. Flow channel 110 can advantageously be used for providing in a region of interest, via VUS transducer 700, for example, contrast agents (e.g., microbubbles, nanodroplets, and the like, which can be used to lower the cavitation threshold). The active cavitation induced by the injection of such contrast agents within the field of the ultrasound acoustic energy produced by VUS transducer 700 can advantageously aid in further improving the therapeutic rate of the swirling characteristic of the ultrasound acoustic energy. The design of VUS transducer 700 shown in FIG. 7 was used for validation of in-vitro intravascular sonothrombolysis of clots aged from 1 hr to 7 days. More than 50% clot lysis rate increasing was observed when using VUS in comparison with conventional non-swirl flow ultrasound transducers. The results of this validation demonstrate that intravascular VUS transducers are suitable for safe and fast lysis of acute and aged blood clots.

According to an example embodiment, VUS transducer 700 can include a small aperture, low frequency, forward-viewing piezoelectric ultrasound transducer array integrated in a 2-lumen 6-French gauge catheter 702 for generating vortex ultrasound (e.g., ultrasound with a swirling characteristic) with sufficient acoustic output (mechanical index, or MI, of about 0.5 to about 1.5) for effective sonothrombolysis. Flow channel 110 of catheter 702 can be used for delivering microbubbles (MBs) and/or nanodroplets (NDs) and a tissue plasminogen activator (t-PA) into the ultrasound field produced by VUS transducer 700. In order to achieve fast and safe thrombolysis of cerebral venous sinus thrombosis (CVST) with minimal doses of t-PA (e.g. <10 μg/mL) and a short treatment time (e.g. <30 min for CVST greater than 3.2 grams), VUS transducer 700 according to this example embodiment comprises a miniaturized 2 by 2 custom transducer array (e.g., having a diameter of about 1.6 mm) with moderate power (e.g., up to 10 W/cm²).

According to this example embodiment, VUS transducer 700 includes a transducer array comprises four (4) piezoelectric stacks 202 each with piezoelectric elements 402, for example two layers of PZT-5A material, each with an aperture of about 0.8 mm×0.8 mm (0.64 mm2) and a longitudinal-excitation-mode resonance frequency of about 1.8 MHz. Piezoelectric elements 402 are mounted onto an epoxy base (e.g., containing air bubbles) with backing layer 706. In embodiments implementing mechanical phase delay shown in FIG. 5B, piezoelectric stacks 202 can include a quarter wavelength (e.g., about 0.21 mm) step in backing layer 706 between neighboring piezoelectric stacks 202 to form the 2×2 helical-patterned transducer array for vortex ultrasound generation. The use of air bubbles in the epoxy base can advantageously be used to enhance the acoustic contrast on the back side of the transducers and the forward ultrasound emission. The total aperture area of the transducer array prototype is about 1.65 mm×1.65 mm (about 2.7 mm²). In some embodiments, the aperture of each transducer in the transducer array can be larger or smaller than 0.8 mm×0.8 mm and the total aperture can be larger or smaller than 1.65 mm×1.65 mm.

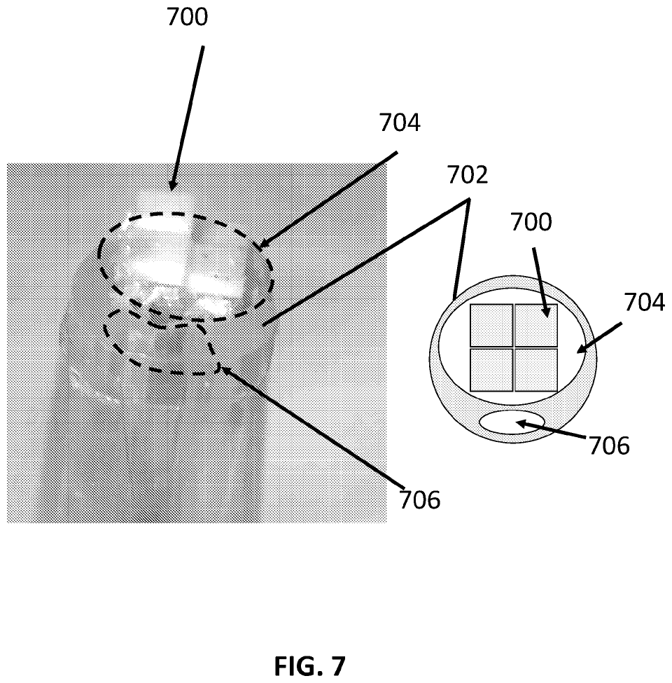

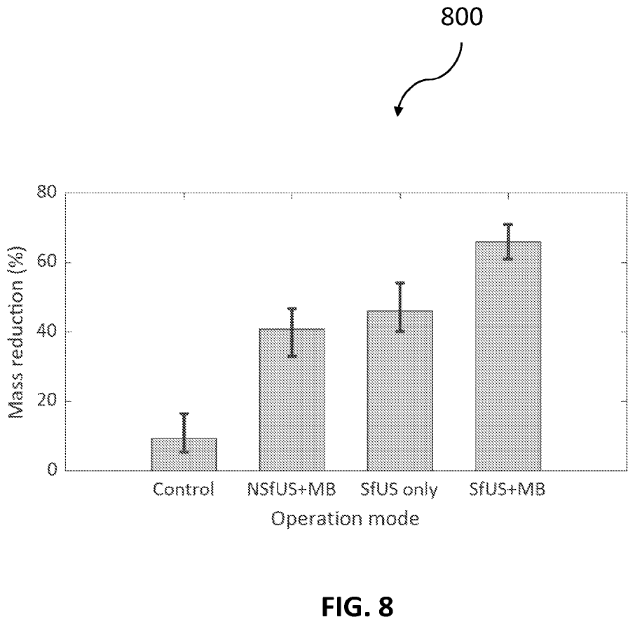

FIG. 8 is a bar chart illustrating the efficacy of using VUS transducer 700 for thrombolysis in-vitro with bovine blood clots, which were up to 7-days old at the time of demonstration. In the results for the example embodiment of VUS transducer 700 shown in FIG. 7, the VUS transducer 700 had an effective lysis rate of over 72% for therapeutic treatment time of about 30 minutes, when combined with microbubble (MB) injection (e.g., through the second lumen of the catheter), which is a statistically significant improvement over a control (e.g., injection of phosphate-buffered saline (PBS) without sonication) and also over the non-vortex ultrasound (NVUS) transducer, operated either with or without microbubble injection.

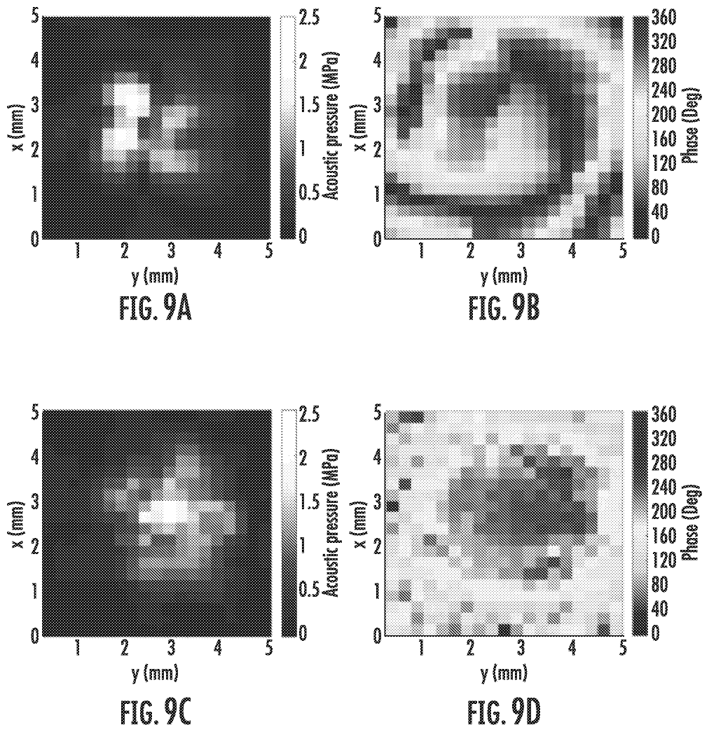

FIGS. 9A and 9B show plots illustrating a measured ultrasound field generated by VUS transducer 700. FIG. 9A shows a plot of the amplitude field, i.e., acoustic pressure, of VUS transducer 700 and FIG. 9B shows a plot of the phase pattern generated by the VUS transducer 700. In this example embodiment, the transducer array is integrated in an 8-French 2-lumen catheter with a flow channel for localized injection of MBs/NDs and lytic agent(s). With 80 Vₚₚ voltage input, the resultant acoustic pressure field (e.g., both amplitude and phase) about 1.5 wavelengths (e.g., about 1.2 mm) away from the transducer array is shown in FIGS. 9A and 9B. A toroidal-like pattern in the pressure amplitude and a spiral pattern in the acoustic phase can be observed, respectively, for the acoustic vortex beam produced using the transducer array. The-6 dB beam (insonation zone) diameter of the vortex ultrasound is around 2.5 mm and is slightly wider along propagation downstream due to diffraction. The peak mechanical index (MI) achieved for the example embodiment of the VUS transducer disclosed herein is around 1.5 in the near-field (<0.5 wavelengths) of the transducer array and is about 0.8 at a distance of about 1.5 wavelengths away, which is sufficient to cause MB/ND mediated cavitation for enhanced sonothrombolysis and is also safe for intravascular operation. In comparison, FIGS. 9C and 9D shows plots of the amplitude field, i.e., acoustic pressure, and phase pattern generated by a transducer array with a flat forward viewing surface. The transducer array with a flat forward viewing surface generates a conventional Gaussian beam profile with the peak pressure amplitude at the center of the beam. The resultant peak pressure amplitude and negative-peak-pressure for a transducer array with a flat forward viewing surface are higher than the vortex ultrasound, but the in-plane pressure gradient is much smaller than the vortex ultrasound field.

Among the advantages associated with the use of vortex ultrasound is caused by the large in-plane pressure gradient that will induce a rotating shear flow in fluids and a significant shear stress in the interacting solids. Using the VUS transducers disclosed herein for sonothrombolysis, the induced shear stress in the blood clots will loosen and break the fibrins, thereby significantly enhancing the sonothrombolysis rate and reduce the required treatment time and lytic agent dose for fast and safe CVST treatment. A rotational shear flow is induced by the vortex ultrasound produced by the VUS transducer; a flat-front transducer array produces a Gaussian wave without inducing any shear flow. The vortex ultrasound, even without optimizing the acoustic parameters, has been demonstrated capable of accelerating the lytic rate (in vitro) by about 65% compared to a traditional Gaussian wave pattern.

According to the VUS transducer design disclosed herein, the acoustic phase delay of each transducer element is determined by the azimuthal polar coordinate of each piezoelectric element of the transducer array. The acoustic phase delay (φ) of an element with an in-plane azimuthal polar coordinate θ is given by φ=lθ to generate vortex ultrasound with topological charge l (a quantity that measures the angular momentum carried by the vortex wave). A larger topological charge is associated with a vortex wave with higher angular momentum and larger aperture. For the 2×2 transducer array in the example embodiment, vortex ultrasound with l=±1 can be excited, which provides the smallest aperture size achievable by vortex ultrasound. In this example embodiment, the acoustic phase delay between adjacent transducers (e.g., in the circumferential direction) is π/2, corresponding to a quarter wavelength shift in forward viewing surfaces between adjacent transducers of the array. By aligning the four elements with a quarter wavelength (e.g., about 0.21 mm for 1.8 MHz) shift between the forward viewing surfaces of adjacent transducers using the epoxy base. A toroidal-like pattern in the amplitude and a spiral pattern in the phase can be produced for a vortex ultrasound according to the example embodiment, despite the asymmetric amplitude field. A slight misalignment of the transducer forward-viewing surfaces can cause an asymmetric acoustic pressure field, which can therefore lead to asymmetric lysis.

Another example embodiment of VUS transducer 700 comprises a transducer array with a flat forward viewing surface and electrical phase delay (e.g., π/2 between adjacent transducers) in the (e.g., AC) voltage input on each transducer of the array, as shown in FIG. 5A. Such an electrical phase delay can be produced using analog all-pass filters or digital control circuits. To achieve a symmetric pressure field, the electrical phase delay can be adjusted for transducer array calibration. An additional advantage of the example embodiment in which an electrical phase delay is used is that the rotation of the helical wavefront of vortex ultrasound can be reversed by reversing the direction of the phase delay between the adjacent transducers, thereby allowing the alternating of shear stress direction in the blood clot, which can further enhance lysis efficiency.

Since, in the example embodiments disclosed herein for use in CVST, the transducer array needs to fit in a 6-French catheter to be sufficiently flexible for guidance into the cerebral venous sinus for CVST, a small aperture is a key feature that must be achieved in the transducer array design. However, the −6 dB beam diameter will increase with a smaller transducer array aperture due to acoustic diffraction. For a transducer array with aperture between about 1.4 mm to about 1.6 mm in diameter that fits in a 6-French catheter, the insonation zone area is around 4×4 mm² at the target distance (e.g., about 1 mm away) with MI of about 0.5-1.5 using a <100 V driving voltage (peak-to-peak) for each transducer.

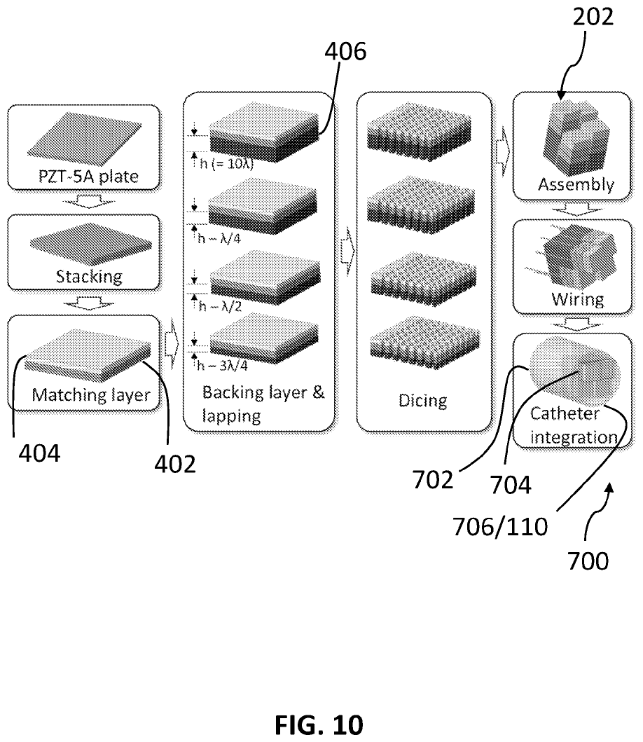

FIG. 10 is a flow chart illustrating an example method of manufacturing a VUS transducer, for example VUS transducer 700, by which transducer array for use in the VUS transducer 700 can be produced. In this embodiment, VUS transducer 700 includes a piezoelectric transducer. Two plates of piezoelectric elements 402 (e.g., PZT-5A, area of about 5 mm×5 mm and thickness of about 200 μm) are bonded together with an adhesive, for example using conductive silver epoxy (e.g., 3022 E-Solder, from Von Roll, Breitenbach, Switzerland) with a thickness of about 20 μm. A quarter-wavelength matching layer 404 made of alumina powder/epoxy bond mixture with an acoustic impedance of ˜7-8 MRayls is then attached at the front side of the piezoelectric plates 402. Air bubble/epoxy composite backing is then deposited onto the rear side of the piezoelectric plates 402, with a thickness of about 6 wavelengths (e.g., about 1.5 mm). Four piezoelectric multi-layers forming piezoelectric elements 402 integrated with matching layer 404 and backing layer 406 have a different aperture height (e.g., a quarter wavelength step, for a four-transducer array) by lapping the backing layer 406 to achieve a prescribed thickness. For producing a transducer array that uses an electrical phase delay, the epoxy backing layers have the same height.

The multi-layered specimens are then diced for the designed element aperture of around 0.6 mm×0.6 mm (e.g., DAD323, from Disco Corp., Tokyo, Japan), producing four multilayered piezoelectric stacks 202 having different aperture heights. The multilayered piezoelectric stacks 202 are bonded together using an electrically nonconductive alumina/epoxy composite, followed by the providing an electrical connection. After fabrication, the impedance spectrum can be, in some embodiments, measured to verify the resonance frequency. To characterize the generated acoustic waveform and pressure output, the transducer array can be mounted on a computer-controlled 3-axis translational stage, and the acoustic pressure field measured by a calibrated hydrophone about 1 mm away from the transducer array. A function generator (e.g., 33250A, from Agilent Tech. Inc., Santa Clara, CA) can be used to transmit a sinusoidal pulse of 10 cycles per 10 ms to an RF power amplifier (e.g., 75A250A, from AR, Souderton, PA). The amplified signal is then fed into VUS transducer 700. In some embodiments, it is then confirmed that the measured pressure and corresponding MI (e.g., about 0.5-1.5) is sufficient to enhance cavitation of MBs/NDs and safe for operation in a designated physiological location (e.g., cerebral venous sinus). Following the characterization of VUS transducer 700, the transducer array is integrated into a 2-lumen flexible catheter 702 having a 6-French diameter; first lumen 704 guides the piezoelectric transducer, and second lumen 706 provides a flow channel 110 for drug and contrast agent delivery. Catheter 702 can be made from, for example, polyethylene, which can advantageously make the 6-French catheter sufficiently flexible for guidance into the designated physiological location (e.g., the cerebral venous sinus).

The optimal input parameters for the VUS transducers disclosed herein with a desired insonation zone can be determined by (e.g., characterized by) initial in vitro tests with MBs/NDs injection. Among the input parameters that can be varied to control operation of the VUS are one or more of the following: peak-to-peak voltage, duty cycle, concentration of MBs/NDs, and injection flow rate of MBs/NDs. For controlling peak-to-peak voltage, the driving voltage is determined by the required acoustic pressure (e.g., MI of about 0.5-1.5), piezo material coercive field, and safety. A peak-to-peak voltage of less than 100V may be used in some instances, but is not limited thereto. For controlling duty cycle, the acoustic pressure, lysis rate, and transducer temperature change can be monitored as (e.g., simultaneously) the duty cycle is varied. The duty cycle can, in some embodiments, be controlled to be less than about 25% to avoid unwanted heating of the VUS transducer, but duty cycles of 25% or more may be used in some instances (e.g., for prescribed periods of time that are shorter than the time needed for therapeutic treatment). For controlling the concentration of MBs/NDs, a concentration may be varied from, for example, about 0 to about 1×10⁹/mL for transducer operation parameters optimization. A concentration of about 1×10⁷/mL is approximately a clinical dose of Definity microbubble contrast agent, as diluted in circulating blood flow, which can be the lowest range of concentrations. A concentration of about 1×10¹⁰/mL is an undiluted microbubble solution, which may also be feasible for use, since it is delivered directly to the blood clot via second lumen 706 of the catheter. The NDs have substantially similar concentrations as MBs. For controlling the flow rate of injection for MBs/NDs, the: flow rate can be varied from about 10 μL/min to about 200 μl/min, which, for example, corresponds to about 100 therapeutic treatments of about a 5 minute duration to stay within the indicated 10 μL/kg clinical doses of Definity contrast agent for a 100 kg patient, but the VUS transducers disclosed herein are not limited to such flow rates in all embodiments.

In some embodiments, second lumen 706 of catheter 702 can be attached to, but formed discrete from, first lumen 704 of the catheter 702, in the form of a microtube. It is advantageous for second lumen 706 of catheter 702, whether having a unitary or discrete construction with first lumen 704, to have an outlet that is oriented so the injected MBs/NDs flow between the transducer array radiation surface and the target clot. The position of VUS transducer 700 can be controlled, for example, by a 3-axis motion controller. An important design consideration for such VUS transducers is the durability thereof, since VUS transducer 700 is expected to operate substantially continuously for a relatively long time (e.g., on the order of about ten(s) of minutes to hours). Thus, the durability with long treatment time (>1-5 hours) for different input conditions is advantageously ensured for VUS transducers disclosed herein.

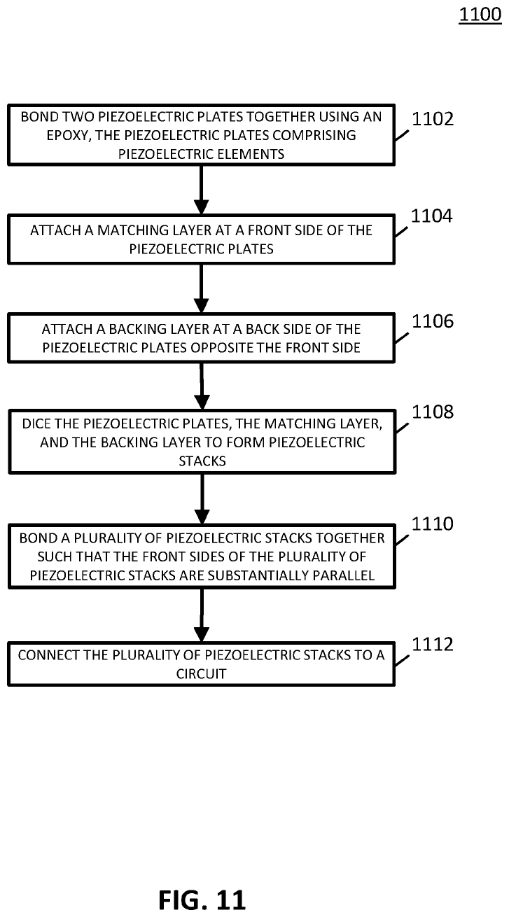

FIG. 11 is a flow diagram illustrating example method 1100 for producing an ultrasound transducer, wherein this example embodiment of the VUS transducer includes a piezoelectric transducer. At step 1102, multiple piezoelectric plates are bonded together using an epoxy, the piezoelectric plates comprising piezoelectric elements. In some embodiments, two piezoelectric plates are bonded together. Alternative poling directions may be applied between adjacent piezoelectric plates in piezoelectric elements 402. The target area may be a blood clot.

At step 1104, a matching layer is attached at a front side of the piezoelectric plates.

At step 1106, a backing layer is attached at a back side of the piezoelectric plates opposite the front side.

At step 1108, the piezoelectric plates, the matching layer, and the backing layer are diced to form piezoelectric stacks. One or more isolation layers including electrically insulating material may be applied to the piezoelectric stacks for a wire connection to electrically connect the piezoelectric stacks in parallel.

At step 1110, a plurality of piezoelectric stacks are bonded together such that the front sides of the plurality of piezoelectric stacks are substantially parallel.

At step 1112, the plurality of piezoelectric stacks are connected to a circuit, wherein the circuit is configured to generate at least one signal to activate the piezoelectric elements, wherein the piezoelectric stacks are arranged to generate ultrasound energy with a swirling movement that produces a mechanical shear stress over a target area within a blood vessel of a patient. The plurality of piezoelectric stacks may include at least three piezoelectric stacks. The piezoelectric elements may be sequentially activated in a clockwise direction or a counterclockwise direction, with respect to a longitudinal axis of the VUS transducer. The piezoelectric elements of each of the plurality of piezoelectric stacks may have sequentially greater height in a clockwise direction or a counterclockwise direction, with respect to a longitudinal axis of the VUS transducer.

The piezoelectric elements of each of the plurality of piezoelectric stacks may have approximately a same height. The circuit may be configured to generate a signal targeting the piezoelectric elements of a select piezoelectric stack of the plurality of piezoelectric stacks. The circuit may be configured to sequentially transmit a signal to each of the plurality of piezoelectric stacks height in a clockwise direction or a counterclockwise direction, with respect to a longitudinal axis of the VUS transducer. The circuit may be configured to reverse the sequential transmission.

Method 1100 may include an optional step of housing the plurality of piezoelectric stacks in a catheter, wherein the catheter comprises a flow channel configured to inject one or more contrast agents within a field of the ultrasound energy emitted from the ultrasound transducer.

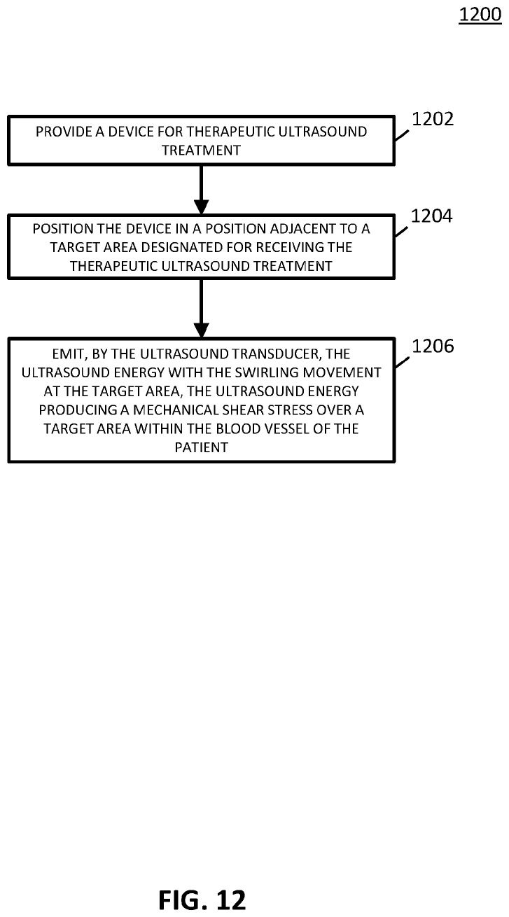

FIG. 12 is a flow diagram illustrating example method 1200 of therapeutic ultrasound treatment. At step 1202, a device for therapeutic ultrasound treatment is provided. The device includes a jacket for insertion within a blood vessel of a patient. The device also includes an ultrasound transducer located within the jacket and comprising a plurality of active elements that are arranged such that, when activated, the ultrasound transducer is configured to generate ultrasound energy with a swirling movement.

The jacket may include a catheter, wherein the catheter comprises a flow channel configured to inject one or more contrast agents within a field of the ultrasound energy emitted from the ultrasound transducer. Each of the plurality of active elements may be a piezoelectric element. The plurality of active elements may be sequentially activated in a clockwise direction or a counterclockwise direction, with respect to a longitudinal axis of the ultrasound transducer. The plurality of active elements may have sequentially greater height in a clockwise direction or a counterclockwise direction, with respect to a longitudinal axis of the ultrasound transducer. The plurality of active elements may have approximately a same height. The device may include a circuit connected to the plurality of active elements, wherein the circuit is configured to generate a signal targeting one or more select active elements of the plurality of active elements. The circuit may be configured to sequentially transmit a signal to one or more select active elements of the plurality of active elements in a clockwise direction or a counterclockwise direction, with respect to a longitudinal axis of the ultrasound transducer. The circuit may be configured to reverse the direction of the sequential transmission.

At step 1204, the device is positioned in a position adjacent to a target area designated for receiving the therapeutic ultrasound treatment.

At step 1206, the ultrasound transducer emits the ultrasound energy with the swirling movement at the target area, the ultrasound energy producing a mechanical shear stress over a target area within the blood vessel of the patient.

The target area may be a blood clot.

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent techniques that would be apparent to one skilled in the art. While the following terms are believed to be well understood by one of ordinary skill in the art, the following definitions are set forth to facilitate explanation of the presently disclosed subject matter.

In describing the presently disclosed subject matter, it will be understood that a number of techniques and steps are disclosed. Each of these has individual benefit and each can also be used in conjunction with one or more, or in some cases all, of the other disclosed techniques.

Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

Following long-standing patent law convention, the terms “a”, “an”, and “the” refer to “one or more” when used in this application, including the claims. Thus, for example, reference to “a component” includes a plurality of such components, and so forth.

Unless otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in this specification and attached claims are approximations that can vary depending upon the desired properties sought to be obtained by the presently disclosed subject matter.

As used herein, the term “about,” when referring to a value or to an amount of a composition, mass, weight, temperature, time, volume, concentration, percentage, etc., is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

The term “comprising”, which is synonymous with “including” “containing” or “characterized by” is inclusive or open-ended and does not exclude additional, unrecited elements or method steps. “Comprising” is a term of art used in claim language which means that the named elements are essential, but other elements can be added and still form a construct within the scope of the claim.

As used herein, the phrase “consisting of” excludes any element, step, or ingredient not specified in the claim. When the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of a claim to the specified materials or steps, plus those that do not materially affect the basic and novel characteristic(s) of the claimed subject matter.

With respect to the terms “comprising”, “consisting of”, and “consisting essentially of”, where one of these three terms is used herein, the presently disclosed and claimed subject matter can include the use of either of the other two terms.

As used herein, the term “and/or” when used in the context of a listing of entities, refers to the entities being present singly or in combination. Thus, for example, the phrase “A, B, C, and/or D” includes A, B, C, and D individually, but also includes any and all combinations and subcombinations of A, B, C and D.

It will be understood that various details of the presently disclosed subject matter may be changed without departing from the scope of the presently disclosed subject matter. Furthermore, the foregoing description is for the purpose of illustration only, and not for the purpose of limitation.

What is claimed is:

1. A device for therapeutic ultrasound treatment, the device comprising:
a jacket for insertion within a blood vessel of a patient; and
an ultrasound transducer located within the jacket and comprising a plurality of active elements that are arranged such that, when activated, the ultrasound transducer is configured to generate ultrasound energy with a swirling movement that produces a mechanical shear stress over a target area within the blood vessel of the patient.

2. The device of claim 1 wherein the jacket comprises a catheter, wherein the catheter comprises a flow channel configured to inject one or more contrast agents within a field of the ultrasound energy emitted from the ultrasound transducer.

3. The device of claim 1 wherein the target area is a blood clot.

4. The device of claim 1 wherein each of the plurality of active elements is a piezoelectric element.

5. The device of claim 1 wherein the plurality of active elements are sequentially activated in a clockwise direction or a counterclockwise direction, with respect to a longitudinal axis of the ultrasound transducer.

6. The device of claim 1 wherein the plurality of active elements have sequentially greater height in a clockwise direction or a counterclockwise direction, with respect to a longitudinal axis of the ultrasound transducer.

7. The device of claim 1 wherein the plurality of active elements have approximately a same height.

8. The device of claim 7 comprising a circuit connected to the plurality of active elements, wherein the circuit is configured to generate a signal targeting one or more select active elements of the plurality of active elements.

9. The device of claim 8 wherein the circuit is configured to sequentially transmit a signal to one or more select active elements of the plurality of active elements in a clockwise direction or a counterclockwise direction, with respect to a longitudinal axis of the ultrasound transducer.

10. The device of claim 9 wherein the circuit is configured to reverse the direction of the sequential transmission.

11. A method of therapeutic ultrasound treatment, the method comprising:
providing a device for therapeutic ultrasound treatment comprising:
a jacket for insertion within a blood vessel of a patient; and
an ultrasound transducer located within the jacket and comprising a plurality of active elements that are arranged such that, when activated, the ultrasound transducer is configured to generate ultrasound energy with a swirling movement;
positioning the device in a position adjacent to a target area designated for receiving the therapeutic ultrasound treatment; and
emitting, from the ultrasound transducer, the ultrasound energy with the swirling movement at the target area, the ultrasound energy producing a mechanical shear stress over a target area within the blood vessel of the patient.

12. The method of claim 11 wherein the jacket comprises a catheter, wherein the catheter comprises a flow channel configured to inject one or more contrast agents within a field of the ultrasound energy emitted from the ultrasound transducer.

13. The method of claim 11 wherein the target area is a blood clot.

14. The method of claim 11 wherein each of the plurality of active elements is a piezoelectric element.

15. The method of claim 11 wherein the plurality of active elements are sequentially activated in a clockwise direction or a counterclockwise direction, with respect to a longitudinal axis of the ultrasound transducer.

16. The method of claim 11 wherein the plurality of active elements have sequentially greater height in a clockwise direction or a counterclockwise direction, with respect to a longitudinal axis of the ultrasound transducer.

17. The method of claim 11 wherein the plurality of active elements have approximately a same height.

18. The method of claim 17 wherein the device comprises a circuit connected to the plurality of active elements, wherein the circuit is configured to generate a signal targeting one or more select active elements of the plurality of active elements.

19. The method of claim 18 wherein the circuit is configured to sequentially transmit a signal to one or more select active elements of the plurality of active elements in a clockwise direction or a counterclockwise direction, with respect to a longitudinal axis of the ultrasound transducer.

20. The method of claim 19 wherein the circuit is configured to reverse the direction of the sequential transmission.
